(19) United States
(12) Patent Application Publication
(10) Pub. No.: US 2008/0154218 A1
(43) Pub. Date: Jun. 26, 2008

(54) Rigid sleeve phacoemulsification needle

(76) Inventors: Mario P. Gomez, St. Louis, MO (US); Ildefonso Gonzalez, St. Charles, MO (US); Dimas Joel Rullan, St. Charles, MO (US)

Correspondence Address: Bausch & Lomb Incorporated, One Bausch & Lomb Place, Rochester, NY, 14604-2701, US

(21) Appl. No.: 11/643,380

(22) Filed: Dec. 21, 2006

(51) Int. Cl. A61M 5/158

(52) U.S. Cl. 604/274; 604/264; 604/46

(57) ABSTRACT

A phacoemulsification needle 10 includes an elongated lumen 12, a hub 14, and a rigid sleeve 16 concentrically formed around at least a majority of the lumen 12. The sleeve 16 is attached to the hub 14. An irrigation fluid path 24 is formed between the sleeve 16 and lumen 12 and a portion of the path 24 is also formed in the hub 14 for carrying irrigation fluid from a source through the path 24 to a patient's eye during surgery.

### BACKGROUND OF THE INVENTION

1. Field of the Invention

The present invention is directed to phacoemulsification needles and more particularly, to phacoemulsification needles having a concentric coaxial rigid sleeve surrounding the needle.

2. Description of Related Art

Sleeves surrounding phacoemulsification needles are well known in the art. Such sleeves generally perform the function of directing the flow of irrigation fluid from an irrigation source into the eye. By directing the irrigation fluid to flow into the eye in a path between the phacoemulsification needle and the sleeve, irrigation fluid may provide a cooling effect to the phacoemulsification needle and conveniently provides aspiration and irrigation through a single incision in the eye. Such coaxial phacoemulsification needles and sleeves are well known. Such sleeves typically are made from pliable materials, such as silicone, and provide sealing of the incision by at least partially collapsing around the phacoemulsification needle. Such collapsing of the sleeve around the needle, while beneficial for sealing the incision site, causes the irrigation fluid flow to be interrupted, if not completely blocked. There have been a number of designs to ensure that fluid flow is maintained, such as providing ribs on the interior of the sleeve or the exterior of the phacoemulsification needle.

The use of such pliable materials for sleeves is effective, but requires incisions that are typically larger than 2 millimeters. It is increasingly desired to perform surgery using an incision size as small as possible.

It is also known to use metal sleeves or other rigid materials to surround a needle. However, such use of rigid materials in the prior art required the use of a sleeve with its own hub for attachment to a handpiece or some other separate attachment scheme for attaching the sleeve to the handpiece. This makes for a larger and more cumbersome needle-sleeve arrangement than is desired, and may also in fact result in frictional contact between the sleeve and the needle which would create undesired heat during surgery and could potentially damage an eye.

Therefore, it would be desirable to provide a rigid sleeve surrounding a needle which is attached to the hub of a phacoemulsification needle for convenient and compact construction.

### BRIEF DESCRIPTION OF THE DRAWINGS

FIG. 1 is a perspective view of a phacoemulsification needle in accordance with the present invention;

FIG. 2 is a cut away elevation view taken along line 2-2 of FIG. 1; and

FIG. 3 is a cut away elevation view of an alternate embodiment of a phacoemulsification needle in accordance with the present invention.

### DETAILED DESCRIPTION OF THE PREFERRED EMBODIMENT

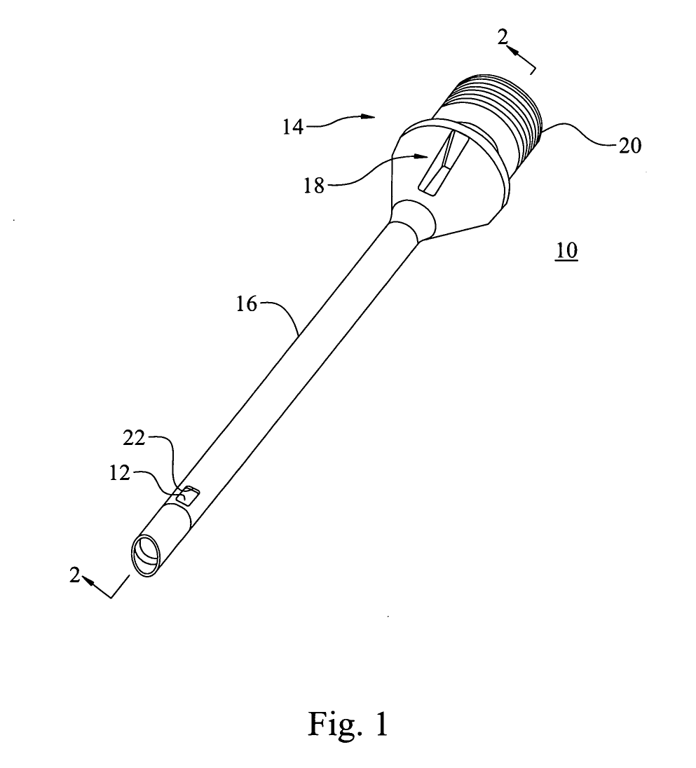

FIG. 1 shows a phacoemulsification needle 10, in accordance with the present invention. Needle 10 includes an elongated lumen shown at 12 having a distal end and a proximal end. A hub, shown generally at 14, is formed on the proximal lumen end for attachment to a handpiece (not shown). A rigid sleeve 16 is concentrically formed around at least a majority of the lumen 12 and the sleeve 16 is attached to the hub 14. An irrigation fluid path (shown in detail below) is formed between the sleeve 16 and the lumen 12 and a portion of the path is also formed in the hub 14, shown generally at 18. Reference numeral 18 generally shows a port or an inlet for carrying irrigation fluid from a source (not shown) through the path to a patient's eye during surgery.

Hub 14 preferably includes an attachment portion 20, such as the threads shown for attachment to a standard phacoemulsification handpiece. Lumen 12, hub 14, and sleeve 16 are all formed preferably of a suitable metal, and more preferably of titanium. By using titanium, it is believed that the entire phacoemulsification needle 10 can be made with a cross-sectional diameter small enough to allow incision sizes of about 1.6 millimeters, but in any case below 2 mm. Such a small incision size is currently desirable and is believed to aid in the recovery of the patient after surgery with fewer optic consequences as compared to a larger incision size.

Preferably sleeve 16 includes a port 22 formed adjacent the distal end of the lumen for providing an exit for the irrigation fluid.

A fluid-tight seal is formed between the lumen and the sleeve at the hub 14. Such fluid-tight seals may be accomplished by laser welding, diffusion bonding, threads, or any other suitable attachment for sealing the sleeve 16 to the hub 14.

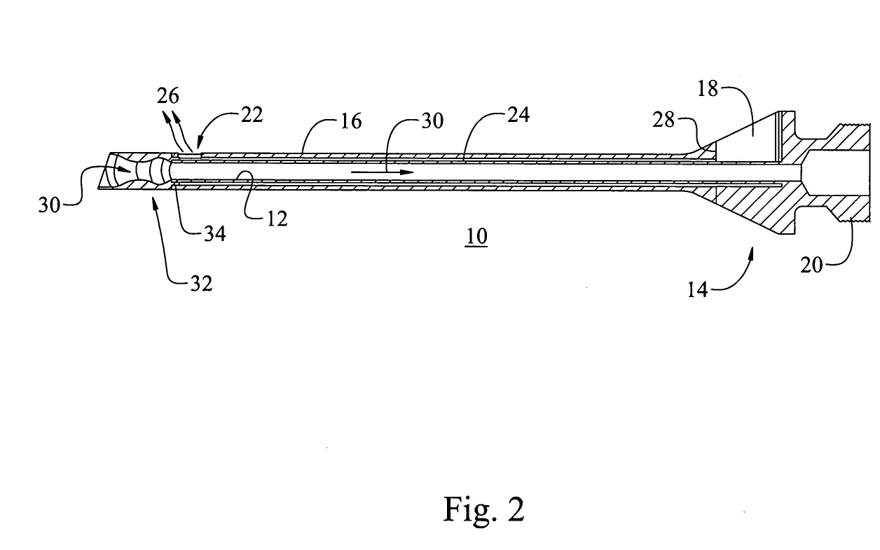

FIG. 2 shows a cross-sectional elevation view of FIG. 1 taken along line 2-2. As can be seen, from inlet 18 to port 22 an irrigation fluid path 24 is formed to allow irrigation fluid to flow from a source not shown through port 22 as shown by arrows 26. In use, a silicone boot, not shown, may be attached to a conventional phaco handpiece similar to flexible phaco sleeves well known in the art. The difference between the boot to be used with the inventive phacoemulsification needle 10 and the prior art, is that the boot would only cover the hub 14 so that fluid could be delivered to inlet 18. When inlet 18 is exposed to the flow of irrigation fluid from a source, obviously the boot would require a liquid-tight seal to be formed around sleeve 16 to prevent or minimize leakage of irrigation fluid. In this way, the phacoemulsification needle 10 may be used with a conventional phacoemulsification handpiece. This is compared to prior art metal sleeves which would require a separate connection with the phacoemulsification handpiece. By attaching the sleeve 16 to hub 18 at weld-joint 28 it is possible to easily ensure that sleeve 16 is parallel to lumen 12 and not cause unwanted frictional contact between the sleeve 16 and the lumen 12.

In use, ophthalmic tissue and fluid is aspirated through lumen 12 by an aspiration pump, not shown, along the path indicated by arrows 30.

In the embodiment shown in FIG. 2, the lumen 12 has a flared distal end 32 of a greater diameter than a diameter of a majority of the lumen 12. The sleeve 16 is attached to the flared distal end 32 of the lumen 12 at joint 34. Joint 34 again is formed preferably by laser welding or diffusion bonding as is known in the art.

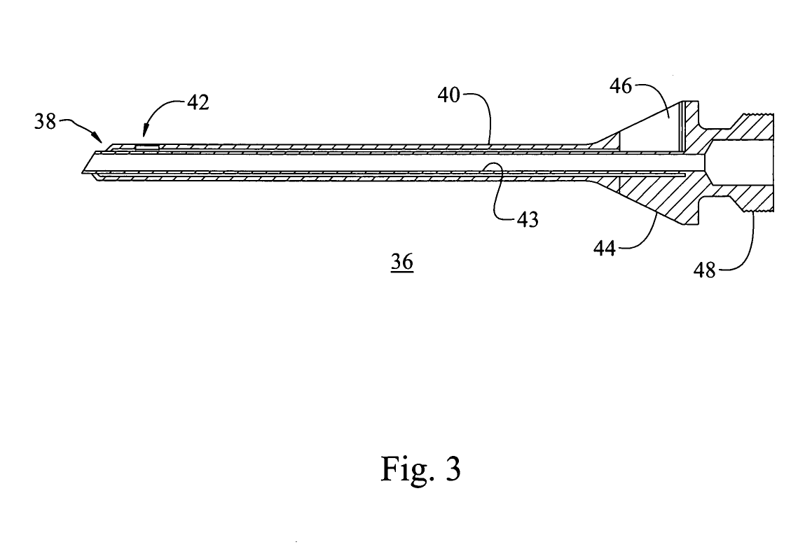

FIG. 3 shows an alternate embodiment in accordance with the present invention. The alternate embodiment is shown in a cross-sectional elevation view similar to that in FIG. 2. Phacoemulsification needle 36 of FIG. 3 differs from phacoemulsification needle 10, in that the distal end 38 does not have a flared end. However, again a fluid-tight seal is preferably formed between the distal end 38 and the sleeve 40, as described above. Sleeve 40 also preferably includes a port 42 similar to that described above with regard to port 22. While a fluid-tight seal at the distal end between the lumen and the sleeve is preferred, it is also possible that no port is formed in the outer sleeve and no seal is formed between the lumen and sleeve, thus allowing irrigation fluid to flow out the end of the sleeve into the eye during surgery. In all other respects FIG. 3 is identical to FIG. 2 in that it includes a lumen 42, a hub 44, a fluid inlet 46, and a phaco handpiece connection section 48.

Thus, there has been shown and described a novel phacoemulsification needle having a rigid sleeve concentrically formed around a lumen. Other embodiments will be apparent to those skilled in the art and are to be considered within the scope of the present invention and claims. Such alternate embodiments may include different connection means for connecting to a phacoemulsification handpiece or a different fluid inlet formed in the hub. In addition, the fluid inlet may be made, such that it directly connects to a specially designed phacoemulsification handpiece so that the boot as described above would not be required to supply irrigation fluid to the inlet of the phacoemulsification needle.

What is claimed:

1. A phacoemulsification needle comprising:
an elongated lumen having a distal end and a proximal end;
a hub formed on the proximal lumen and for attachment to a handpiece;
a rigid sleeve concentrically formed around at least a majority of the lumen and wherein the sleeve is attached to the hub; and
wherein an irrigation fluid path is formed between the sleeve and lumen and a portion of the path is also formed in the hub for carrying irrigation fluid from a source through the path to a patient's eye during surgery.

2. The needle of claim 1 wherein, the lumen, hub, and sleeve are formed of metal.

3. The needle of claim 2, wherein the metal is titanium.

4. The needle of claim 1, wherein the sleeve includes a port formed adjacent the distal end for providing an exit for the irrigation fluid.

5. The needle of claim 1, wherein a fluid-tight seal is formed between a distal end of the sleeve and the lumen.

6. The needle of claim 1, wherein the lumen has a flared distal end of a greater diameter of a majority of the lumen and wherein the sleeve is attached to the flared distal end of the lumen.
